(19) United States
(12) Patent Application Publication
(10) Pub. No.: US 2010/0299991 A1
(43) Pub. Date: Dec. 2, 2010

(54) Hydroxymethylfurfural Ethers from Sugars and Olefins

(75) Inventor: Gerardus Johannes Maria Gruter, Heemstede (NL)

Correspondence Address: HOFFMANN & BARON, LLP, 6900 JERICHO TURNPIKE, SYOSSET, NY, 11791, US

(73) Assignee: FURANIX TECHNOLOGIES B.V., Amsterdam (NL)

(21) Appl. No.: 12/676,745

(22) PCT Filed: Sep. 5, 2008
(86) PCT No.: PCT/EP08/07410

§ 371 Date: Apr. 22, 2010

(30) Foreign Application Priority Data
Sep. 7, 2007 (EP) 07075770.3

(51) Int. Cl. C10L 1/185; C07D 307/58

(52) U.S. Cl. 44/351; 549/479

(57) ABSTRACT

The current invention provides a method for the manufacture of an ether of 5-hydroxymethylfurfural by reacting a hexose-containing starting material with an olefin in the presence of an acid catalyst

### CROSS-REFERENCE TO RELATED APPLICATIONS

This application is the National Stage of International Application No. PCT/EP2008/007410, filed Sep. 5, 2008, which claims priority to European Application No. 07075770.3, filed Sep. 7, 2007, the entire contents of each of which are incorporated by reference herein.

### FIELD OF THE INVENTION

The present invention concerns a method for the manufacture of an ether of 5-hydroxymethylfurfural (5-(hydroxymethyl)-2-furaldehyde, or HMF) from biomass.

### BACKGROUND OF THE INVENTION

Fuel, fuel additives and various chemicals used in the petrochemical industry are derived from oil, gas and coal, all finite sources. Biomass, on the other hand, is considered a renewable source. Biomass is biological material (including biodegradable wastes) which can be used for the production of fuels or for industrial production of e.g. fibres, chemicals or heat. It excludes organic material which has been transformed by geological processes into substances such as coal or petroleum.

Production of biomass derived products for non-food applications is a growing industry. Bio based fuels are an example of an application with strong growing interest. Biomass contains sugars (hexoses and pentoses) that may be converted into value added products. Current biofuel activities from sugars are mainly directed towards the fermentation of sucrose or glucose into ethanol or via complete breakdown via Syngas to synthetic liquid fuels.

EP 0641 854 describes the use of fuel compositions comprising of hydrocarbons and/or vegetable oil derivatives containing at least one glycerol ether to reduce particulate matter emissions.

More recently, the acid catalysed reaction of fructose has been re-visited, creating HMF as an intermediate of great interest. Most processes investigated have the disadvantage that HMF is not very stable at the reaction conditions required for its formation. Fast removal from the water-phase containing the sugar starting material and the acid catalyst has been viewed as a solution for this problem. Researchers at the University of Wisconsin-Madison have developed a process to make HMF from fructose. HMF can be converted into monomers for plastics, petroleum or fuel extenders, or even into fuel itself. The process by prof. James Dumesic and co-workers first dehydrates the fructose in an aqueous phase with the use of an acid catalyst (hydrochloric acid or an acidic ion-exchange resin). Salt is added to salt-out the HMF into the extracting phase. The extracting phase uses an inert organic solvent that favours extraction of HMF from the aqueous phase. The two-phase process operates at high fructose concentrations (10 to 50 wt %), achieves high yields (80% HMF selectivity at 90% fructose conversion), and delivers HMF in a separation-friendly solvent (DUMESIC, James A, et al. “Phase modifiers promote efficient production of Hydroxymethylfurfural from fructose”. Science. 30 juni 2006, vol. 312, no. 5782, p. 1933-1937). Although the HMF yields from this process are interesting, the multi-solvent process has cost-disadvantages due to the relatively complex plant design and because of the less than ideal yields when cheaper and less reactive hexoses than fructose, such as glucose or sucrose, are used as a starting material. HMF is a solid at room temperature which has to be converted in subsequent steps to useful products. Dumesic has reported an integrated hydrogenolysis process step to convert HMF into dimethylfuran (DMF), which is assumed to be an interesting gasoline additive.

In WO 2006/063220 a method is provided for converting fructose into 5-ethoxymethylfurfural (EMF) at 60° C., using an acid catalyst either in batch during 24 hours or continuously via column elution during 17 hours. Applications of EMF were not discussed.

Nonetheless, there remains an interest in a much more efficient and faster method for the manufacture of HMF ethers without the formation of by-products and uncontrolled degradation and without the restriction of using the ethanol reagent as the reaction solvent. The inventors have set out to overcome this shortfall.

Surprisingly, the inventors have found that ethers of HMF may be produced in a reasonable yield from hexose containing feedstock, with reduced levels of by-product formation and in a manner that does not require cumbersome process measures (such as 2-phase systems) or lengthy process times.

### SUMMARY OF THE INVENTION

Accordingly, the current invention provides a method for the manufacture of an ether of 5-hydroxymethylfurfural by reacting a hexose-containing starting material with an olefin in the presence of an acid catalyst.

When the reaction product of the above method is used as such or when it is used as an intermediate for a subsequent conversion the selectivity of the reaction is preferably high as the product is preferably pure. However, when the reaction product of the above method is used as a fuel, a fuel additive or as a fuel or a fuel additive intermediate, the reaction product does not necessarily need to be pure. Indeed, in the preparation of fuel and fuel additives from biomass, which in itself is a mixture of various monosaccharides, disaccharides and polysaccharides, the reaction product may contain non-interfering components such as levulinic acid derivatives and/or derivatives of pentoses and the like. For ease of reference, however, the method and the reaction product are described in terms of the reaction of a hexose-containing starting material, resulting in an ether of HMF.

The current invention also provides for the use of the reaction product made according to the present invention as fuel or as fuel additive. Fuels for blending with the product of the present invention include but are not limited to gasoline and gasoline-ethanol blends, kerosene, diesel, biodiesel (refers to a non-petroleum-based diesel fuel consisting of short chain alkyl (methyl or ethyl) esters, made by transesterification of vegetable oil, which can be used (alone, or blended with conventional petrodiesel) in unmodified diesel-engine vehicles), Fischer-Tropsch liquids (for example obtained from GTL, CTL or BTL gas-to-liquids/coal-to-liquids/biomass to liquids processes), diesel-biodiesel blends and green diesel and blends of diesel and/or biodiesel with green diesel (green diesel is a hydrocarbon obtained by hydrotreating biomass derived oils, fats, greases or pyrolysis oil; see for example the UOP report OPPORTUNITIES FOR BIORENEWABLES IN OIL REFINERIES FINAL TECHNICAL REPORT, SUBMITTED TO: U.S. DEPARTMENT OF ENERGY (DOE Award Number: DE-FG36-05GO15085). The product is a premium diesel fuel containing no sulfur and having a cetane number of 90 to 100). Fuels for blending with the product of the present invention may also include one or more other derivatives of furan and tetrahydrofuran. The invention also provides a fuel composition comprising a fuel element as described above and the reaction product made according to the present invention.

### DETAILED DESCRIPTION OF THE PREFERRED EMBODIMENTS

Biomass resources are well known. The components of interest in biomass are the mono-, di- or polysaccharides (hereinafter referred to as hexose-containing starting material). Suitable 6-carbon monosaccharides include but are not limited to fructose, glucose, galactose, mannose, and their oxidized, reduced, etherified, esterified and amidated derivatives, e.g. aldonic acid or alditol, with glucose being the most abundant, the most economic and therefore the most preferred monosaccharide, albeit less reactive than fructose. On the other hand, the current inventors have also succeeded to convert sucrose, which is also available in great abundance. Other disaccharides that may be used include maltose, cellobiose and lactose. The polysaccharides that may be used include cellulose, inulin (a polyfructan), starch (a polyglucan) and hemi-cellulose. The polysaccharides and disaccharides are converted into their monosaccharide component(s) and dehydrated during the manufacture of the 5-HMF ether.

The olefin used in the method of the current invention is preferably an olefinically unsaturated compound that is susceptible to electrophilic attack. It would thus appear that concurrent with the dehydration of the monosaccharide, a hydro-alkoxy-addition occurs. The addition of alcohols and phenols to the double bond of an olefinically unsaturated compound is discussed in Chapter 15 of Advanced Organic Chemistry, by Jerry March, and in particular under reaction 5-4. (3^{rd} ed., © 1985 by John Wiley & Sons, pp. 684-685). This reference book, however, provides no information on the preparation of renewable liquid fuel (additives). Surprisingly, the in-situ preparation of the HMF is not hampered by the hydro-alkoxy-addition.

Preferred olefins contain 4 carbon atoms or more. Ethylene and propylene are also possible but will be very slow to react, whereas isobutylene has been found to be very useful. Indeed, preferred olefins are cycloolefins and substituted iso olefins such as isobutene, 2-methyl-2-butene, 2-methyl-1-butene, 2-methyl-1-pentene, 2-methyl-2-pentene, 3-methyl-2-pentene, 2-ethyl-1-butene, 2,3-dimethyl-1-butene, 2,3-dimethyl-2-butene, 1-methylcyclopentene, the C7 iso olefins and similar C8 and higher olefins. Also suitable are dienes such as butadiene and isoprene and terpenes such as pinene and limonene. These olefins may be represented by the general formulae

HRC═CR′R″ H2C═CR′R″

wherein each R, R′ and R″ independently represents alkyl, aralkyl and alkenyl group which may be linear, branched or cyclic with up to 7 carbon atoms and which may contain one or two heteroatoms, or R and R′ jointly represent an alkenyl group with up to 7 carbon atoms and which may contain one or two heteroatoms. Of these, substituted olefins having up to 8 carbon atoms in total are preferred, with isobutylene being most preferred. The iso olefins used as reagents are generally used in mixture with other hydrocarbons of similar boiling points. For instance isobutene feedstocks usually are C4 cuts from Fluid Catalytic Cracking (FCC) plants, from Steam Cracking plants or from field butanes Dehydrogenation/Isomerization plants. Depending on their origin, these C4 cuts usually contain between 20 and 50 wt % isobutene. The etherification reaction is highly selective so that nearly only the isoolefins are converted to ethers.

Another class of olefins found to be suitable are cyclic olefins, such as cyclopentene, cyclohexene and alkyl-substituted derivatives thereof. These olefinically unsaturated compounds may contain elements other than carbon, referred to as heteroatoms above, preferably oxygen, provided the olefinically unsaturated bond remains susceptible to electrophilic attack and provided that the other functional groups are compatible with the acid catalysed dehydration reactions and with the hydrolytic cleavage reactions. An example of a hetero-substituted cyclic olefin is dihydropyran, which forms the tetrahydropyranyl ether of HMF.

The amount of olefin used during the manufacture of the HMF ether is preferably at least equimolar on the hexose content of the feedstock, but typically is used in much greater excess. Indeed, the olefin (such as dihydropyran) may be used as solvent or co-solvent. In such a case, a sufficient amount of olefin is present to form the HMF ether. The catalyst is preferably selected such that the olefins are not reacting to dimers, oligomers and or polymers.

The acid catalyst in the method of the present invention can be selected from amongst (halogenated) organic acids, inorganic acids, Lewis acids, ion exchange resins and zeolites or combinations and/or mixtures thereof. It may be a homogeneous catalyst, but heterogeneous catalysts (meaning solid) are preferred for purification reasons. The HMF ethers can be produced with a protonic, Brønsted or, alternatively, a Lewis acid or with catalysts that have more than one of these acidic functionalities.

The protonic acid may be organic or inorganic. For instance, the organic acid can be selected from amongst oxalic acid, levulinic acid, maleic acid, trifluoro acetic acid (triflic acid), methanesulphonic acid or para-toluenesulphonic acid. Alternatively, the inorganic acid can be selected from amongst (poly)phosphoric acid, sulphuric acid, hydrochloric acid, hydrobromic acid, nitric acid, hydroiodic acid, optionally generated in situ.

Certain salts may be used as catalyst, wherein the salt can be any one or more of (NH₄)₂SO₄/SO₃, ammonium phosphate, pyridinium chloride, triethylamine phosphate, pyridinium salts, pyridinium phosphate, pyridinium hydrochloride/hydrobromide/perbromate, DMAP, aluminium salts, Th and Zr ions, zirconium phosphate, Sc and lanthanide ions such as Sm and Y as their acetate or trifluoroactate (triflate) salt, Cr-, Al-, Ti-, Ca-, In-ions, ZrOCl₂, VO(SO₄)₂, TiO₂, V-porphyrine, Zr-, Cr-, Ti-porphyrine.

Lewis acids selected as dehydration catalyst can be any one of ZnCl₂, AlCl₃, BF₃.

Ion exchange resins can be suitable dehydration catalysts. Examples include Amberlite™ and Amberlyst™, Diaion™ and Levatit™. Other solid catalyst that may be used include natural clay minerals, zeolites, supported acids such as silica impregnated with mineral acids, heat treated charcoal, metal oxides, metal sulfides, metal salts and mixed oxides and mixtures thereof. If elevated reactions temperatures are used, as defined hereafter, then the catalyst should be stable at these temperatures.

An overview of catalysts that may be used in the method of the current invention may be found in Table 1 of the review article prepared by Mr. Lewkowski: “Synthesis, chemistry and applications of 5-hydroxymethylfurfural and its derivatives” Arkivoc. 2001, p. 17-54.

The amount of catalyst may vary, depending on the selection of catalyst or catalyst mixture. For instance, the catalyst can be added to the reaction mixture in an amount varying from 0.01 to 40 mole % drawn on the hexose content of the biomass resource, preferably from 0.1 to 30 mole %, more preferably from 1 to 20 mole %.

In the preferred embodiment, the catalyst is a heterogeneous catalyst.

The temperature at which the reaction is performed may vary, but in general it is preferred that the reaction is carried out at a temperature from 50 to 300 degrees Celsius, preferably from 50 to 200 degrees Celsius, more preferably from 75 to 175 degrees Celsius. In general, temperatures higher than 300 are less preferred as the selectivity of the reaction reduces and as many by-products occur, inter alia caramelisation of the sugar. Also, the hydro-alkoxy-addition reaction is most efficient at a temperature between 40 and 160 degrees Celsius, more preferably between 60 and 120 degrees Celsius, most preferably at a temperature around 90 degrees Celsius. Performing the reaction below the lowest temperature is also less preferable because of the slow reaction speed. The reaction of the invention can also be carried out in a system with 2 reactors in series, whereby the dehydration step and the hydro-alkoxy-addition step are carried out in the first and second reactor at higher and lower temperature, respectively. In other words, the reaction may be performed in a single reactor, at a temperature from 40 to 160 degrees Celsius, preferably from 60 to 120, more preferably around 90 degrees Celsius or in two reactors, where in the first reactor the dehydration is performed at a temperature from 50 to 300 degrees Celsius, preferably from 50 to 200 degrees Celsius, more preferably from 100 to 200 degrees Celsius and where in the second reactor the olefin is added for the hydro-alkoxy-addition at a temperature from 40 to 160 degrees Celsius, preferably from 60 to 120 degrees Celsius, more preferably around 90 degrees Celsius. If the reactions are carried out above the boiling temperature of water, then the reactions are preferably carried out under pressure, e.g., 10 bar nitrogen or higher.

The hexose-containing starting material is typically dissolved or suspended in a solvent system which can also be the olefin reactant, in order to facilitate the reaction. The solvent system may be one or more selected from the group consisting of water, sulfoxides, preferably DMSO, ketones, preferably methyl ethylketone, methylisobutylketone and acetone, ethylene glycol ethers, preferably diethyleneglycol dimethyl ether (diglyme) or the reactant olefin. Also so-called ionic liquids may be used. The latter refers to a class of inert ionic compounds with a low melting point, which may therefore be used as solvent. Examples thereof include e.g., 1-H-3-methyl imidazolium chloride, discussed in “Dehydration of fructose and sucrose into 5-hydroxymethylfurfural in the presence of 1-H-3-methyl imidazolium chloride acting both as solvent and catalyst”, by Claude Moreau et al, Journal of Molecular Catalysis A: Chemical 253 (2006) 165-169.

The amount of solvent is preferably sufficient to dissolve or suspend the starting material and to limit undesired side-reactions.

The method of the current invention may be carried out in a batch process or in a continuous process, with or without recycle of (part of) the product stream to control the reaction temperature (recycle via a heat exchanger). For instance, the method of the invention can be performed in a continuous flow process. In such method, homogenous catalysts may be used and the residence time of the reactants in the flow process is between 0.1 second and 10 hours, preferably from 1 second to 1 hours, more preferably from 5 seconds to 20 minutes.

Alternatively, the continuous flow process may be a fixed bed continuous flow process or a reactive (catalytic) distillation process with a heterogeneous acid catalyst. To initiate or regenerate the heterogeneous acid catalyst or to improve performance, an inorganic or organic acid may be added to the feed of the fixed bed or reactive distillation continuous flow process. In a fixed bed process, the liquid hourly space velocity (LHSV) can be from 1 to 1000, preferably from 5 to 500, more preferably from 10 to 250 and most preferably from 25 to 100 min⁻¹.

The above process results in a stable HMF ether, which can then be used as such or be converted into a further derivative before being used as fuel and/or as fuel additive. The HMF ethers of the invention can also be used as or can be converted to compounds that can be used as solvent, as monomer in a polymerization (such as 2,5-furan dicarboxylic acid or FDCA), as fine chemical or pharmaceutical intermediate, or in other applications. Oxidation of 5-(tertbutoxymethyl)furfural using an appropriate catalyst under appropriate conditions such as for example described for p-xylene with a NHPI/Co(OAc)₂/MnOAc)₂ catalyst system in Adv. Synth. Catal. 2001, 343, 220-225 or such as described for HMF with a Pt/C catalyst system at pH<8 in EP 0 356 703 or or such as described for HMF with a Pt/C catalyst system at pH>7 in FR 2 669 634, all with air as an oxidant, resulted in the formation of 2,5-furan dicarboxylic acid (FDCA).

The invention further concerns the use of the HMF ethers prepared by the method of the current invention as fuel and/or as fuel additive. Of particular interest is the use of the ethers in diesel, biodiesel or “green diesel”, given its (much) greater solubility therein than ethanol. Conventional additives and blending agents for diesel fuel may be present in the fuel compositions of this invention in addition to the above mentioned fuel components. For example, the fuels of this invention may contain conventional quantities of conventional additives such as cetane improvers, friction modifiers, detergents, antioxidants and heat stabilizers, for example. Especially preferred diesel fuel formulations of the invention comprise diesel fuel hydrocarbons and HMF ether as above described together with peroxidic or nitrate cetane improvers such as ditertiary butyl peroxide, amyl nitrate and ethyl hexyl nitrate for example.

The addition of the HMF ether of the invention to diesel fuel results in similar NOₓ numbers and a slight increase in CO emissions; however, the addition of sufficient amounts of cetane improvers can be utilized to reduce the NOₓ and CO emissions well below the base reference fuel.

Examples are enclosed to illustrate the method of the current invention and the suitability of the products prepared therefrom as fuel. The examples are not meant to limit the scope of the invention.

### Example 1

### Single Step 5-(tert-butoxymethyl)furfural (tBMF) Formation

In a 7.5 ml batch reactor, 0.053 mmol fructose dissolved in 1 mL diglyme/water 90/10 v/v, was reacted with 0.3 mmol isobutene and 9 mg Bentonite, H2SO4, Sc(III) triflate, Sm(III) triflate, Y(III) triflate and dry Amberlyst 36 catalyst at a temperature of 150 degrees Celsius in for 1 hour. In all experiments 2-5% formation of 5-(tert-butoxymethyl)furfural (tBMF) was detected by HPLC analysis (with UV detector). Mass spectrometry confirmed the formation of tBMF.

### Example 2

### Two Step 5-(tert-butoxymethyl)furfural (tBMF) Formation

In a 7.5 ml batch reactor, 0.053 mmol fructose in 1 mL diglyme/water 90/10 v/v, was reacted for 1 hour at a temperature of 150 degrees Celsius with 9 mg Bentonite as the acid catalyst. After one hour, the reactor was rapidly cooled to 90 degrees Celsius, after which 0.3 mmol isobutene was added and stirring was continued for 4 hours. Two main furan peaks were observed in the UV spectrum during HPLC analysis. Mass spectrometry identified these products as HMF (20%), and tBMF (25%).

### Example 3

### Diesel Fuel Applications

### Fuel Solubility

Fuel solubility is a primary concern for diesel fuel applications. Not all highly polar oxygenates have good solubility in the current commercial diesel fuels. Results show that in the 5 vol %, in the 25 vol % and in the 40 vol % blends of tBMF with commercial diesel, both liquid blend components are completely miscible. In a comparative set of experiments it was shown that ethoxymethylfurfural (EMF) is completely miscible in a 5 vol % blend with commercial diesel, but that phase separation occurs with the 25 vol % and with the 40 vol % blends of EMF and diesel.

### Cetane Number

Oxygenated fuel additives may reduce the natural cetane number of the base diesel fuel. A 0.1 vol % blend of tBMF with additive free diesel fuel was prepared at an outside laboratory for cetane determination according to an ASTM D 6890 certified method. While the reference additive-free diesel showed an average cetane number of 52.5, surprisingly, the 0.1 vol % tBMF blend showed an increase with 0.5 to an average cetane number of 53.0.

### Oxidation Stability

Likewise, oxygenated fuel additives, certainly when containing an aldehyde functional group, often reduce the oxidation stability of the base diesel fuel. A 0.1 vol % blend of tBMF with additive free diesel fuel was prepared at an outside laboratory for oxidation stability determination according to NF en ISO 12205 certified methods. Surprisingly, both the reference additive-free diesel and the 0.1 vol % tBMF blend showed the same oxidation stability, indicating that the oxygenated tMBF added to an additive free diesel base fuel does not decrease the oxidation stability of the blend relative to the pure base diesel.

### Example 4

### Emission Engine Testing

In a D9B diesel engine of a citroen Berlingo test car, comparative testing is performed with normal commercial diesel as a fuel and the same commercial diesel to which 25 vol. % 5-(t-butoxymethyl)furfural (tBMF) was added, respectively. tBMF is added as a liquid and does not yield any mixing or flocculation problems up to a 40 vol % blend ratio. The engine is run stationary with regular diesel initially, after which the fuel supply is switched to the 40 vol % tBMF-diesel blend.

During stationary operation with the commercial diesel fuel and with the 25 vol % tBMF blend, the following measurements were made: total particulate matter, volume, O₂, CO, CO₂, NOₓ (NO+NO₂) and total hydrocarbons.

Total particulate matter was sampled according to NEN-EN 13284-1

Particle size distribution was sampled according to VDI 2066-5

Volume was measured according to ISO 10780

Gases were sampled according to ISO 10396

O₂, CO and CO₂ were analysed according to NEN-ISO 12039

NOₓ (NO+NO₂) was analysed according to NEN-ISO 10849

Total hydrocarbons were analysed according to NEN-EN 13526.

TABLE 1
gas analysis results of 100% commercial diesel fuel.

| | | Average | | |
|---|---|---|---|---|
| Experiment | Component | Concentration | | Emission |
| 1 | CO | 191 | mg/Nm³ | 13 g/h |
| | CO₂ | 2.4% | v/v | — |
| | O₂ | 17.8% | v/v | — |
| | TOC (C₃H₈) | 29 | mg/Nm³ | 2 g/h |
| | NOₓ | 323 | mg/Nm³ | 21 g/h |

TABLE 2
particulate matter results of 100% commercial diesel fuel.

| | Volume | | Total particulate matter | | Particle size |
|---|---|---|---|---|---|
| Experi- | Actual | Normal | Concentration | Emission | PSD <10 μm |
| ment | [m3/h] | [Nm3/h] | [mg/Nm3] | [g/h] | [%] |
| 1 | 80 | 60 | 6.1 | | 98.5 |

TABLE 3
gas analysis results of blend of commercial diesel with 25 vol % tBMF.

| | | Average | | |
|---|---|---|---|---|
| Experiment | Component | Concentration | | Emission |
| 2 | CO | 243 | mg/Nm³ | 16 g/h |
| | CO₂ | 2.5% | v/v | — |
| | O₂ | 17.7% | v/v | — |
| | TOC (C₃H₈) | 40 | mg/Nm³ | 3 g/h |
| | NOₓ | 333 | mg/Nm³ | 22 g/h |

TABLE 4
particulate matter results of blend of
commercial diesel with 25 vol % tBMF.

| | Volume | | Total particulate matter | | Particle |
|---|---|---|---|---|---|
| Experi- | Actual | Normal | Concentration | Emission | PSD <10 μm |
| ment | [m3/h] | [Nm3/h] | [mg/Nm3] | [g/h] | [%] |
| 3b (**) | 80 | 60 | 5.1 | | 100 |

### REFERENCES

DUMESIC, James A, et al. “Phase modifiers promote efficient production of Hydroxymethylfurfural from fructose”. Science. 30 Jun. 2006, vol. 312, no. 5782, p. 1933-1937.
WO 2006/063220
Chapter 15 of Advanced Organic Chemistry, by Jerry March, and in particular under reaction 5-4. (3^{rd} ed., © 1985 by John Wiley & Sons, pp. 684-685).
LEWKOWSKI, Jaroslaw. Synthesis, chemistry and applications of 5-hydroxymethylfurfural and its derivatives. Arkivoc. 2001, p. 17-54.
MOREAU, Claude, et al. “Dehydration of fructose and sucrose into 5-hydroxymethylfurfural in the presence of 1-H-3-methyl imidazolium chloride acting both as solvent and catalyst”, Journal of Molecular Catalysis A: Chemical 253 (2006) p. 165-169.
EP 0641 854
UOP report OPPORTUNITIES FOR BIORENEWABLES IN OIL REFINERIES FINAL TECHNICAL REPORT, SUBMITTED TO: U.S. DEPARTMENT OF ENERGY (DOE Award Number: DE-FG36-05GO15085))
Adv. Synth. Catal. 2001, 343, 220-225
EP 0 356 703
FR 2 669 634

1. Method for the manufacture of an ether of 5-hydroxymethylfurfural by reacting a hexose-containing starting material with an olefin in the presence of an acid catalyst.

2. Method according to claim 1, wherein the olefin contains 4 or more carbon atoms and is selected from one or more of the group comprising iso-olefins, cyclo-olefins, terpenes, dienes and cyclodienes.

3. Method according to claim 2, wherein the olefin is represented by at least one of the general formulae (I) or (II):
HRC═CR′R″ (I)
H2C═CR′R″ (II)
wherein each R, R′ and R″ independently represents an alkyl, aralkyl and alkenyl group which may be linear, branched or cyclic with up to 7 carbon atoms and which may contain one or two heteroatoms, or R and R′ jointly represent an alkenyl group with up to 7 carbon atoms and which may contain one or two heteroatoms.

4. Method according to claim 3, wherein the olefin is selected from one or more of the group comprising isobutene, cyclopentene, cyclohexene, norbornene pinene, limonene and dihydropyran, preferably selected from isobutene and dihydropyran.

5. Method according to claim 1, wherein the acid catalyst is selected from the group consisting of homogeneous or heterogeneous acids selected from solid organic acids, inorganic acids, salts, Lewis acids, ion exchange resins, zeolites or mixtures and/or combinations thereof.

6. Method according to claim 5, wherein the acid is a solid Brønsted acid.

7. Method according to claim 5, wherein the acid is a solid Lewis acid.

8. Method according to claim 1, wherein the reaction is performed in a single reactor, at a temperature from 40 to 160 degrees Celsius, preferably from 60 to 120, more preferably around 90 degrees Celsius.

9. Method according to claim 1, wherein the reaction is performed in two reactors, where in the first reactor the dehydration is performed at a temperature from 50 to 300 degrees Celsius, preferably from 50 to 200 degrees Celsius, more preferably from 75 to 175 degrees Celsius and where in the second reactor the olefin is added for the hydro-alkoxy-addition at a temperature from 40 to 160 degrees Celsius, preferably from 60 to 120 degrees Celsius, more preferably around 90 degrees Celsius.

10. Method according to claim 1, wherein a hexose-containing starting material is used and wherein the hexose starting material is selected from the group consisting of
starch, amylose, galactose, cellulose, hemi-cellulose,
glucose-containing disaccharides such as sucrose, maltose, cellobiose, lactose, preferably glucose-containing disaccharides, more preferably sucrose,
glucose or fructose.

11. Method according to claim 10, wherein the hexose-containing starting material is selected from the group of sucrose, glucose, fructose or mixtures thereof.

12. Method according to claim 1, performed in the presence of a solvent, wherein the solvent or solvents are selected form the group consisting of water, sulfoxides, preferably DMSO, ketones, preferably methyl ethylketone, ionic liquids, methylisobutylketone and/or acetone, esters, ethers, preferably ethylene glycol ethers, more preferably diethyleneglycol dimethyl ether (diglyme) or the reactant olefin and mixtures thereof.

13. Method according to claim 1, wherein the method is performed in a continuous flow process.

14. Method according to claim 13, wherein the residence time in the flow process is between 0.1 second and 10 hours, preferably from 1 second to 1 hours, more preferably from 5 seconds to 20 minutes.

15. Method according to claim 14, wherein the continuous flow process is a fixed bed continuous flow process.

16. Method according to claim 15, wherein the fixed bed comprises a heterogeneous acid catalyst.

17. Method according to claim 16, wherein the continuous flow process is a reactive distillation or a catalytic distillation process.

18. Method according to claim 16, wherein in addition to a heterogeneous acid catalyst, an inorganic or organic acid catalyst is added to the feed of the fixed bed or catalytic distillation continuous flow process.

19. Method according to claim 15, wherein the liquid hourly space velocity (“LHSV”) is from 1 to 1000.

20. Use of the ether produced by the method of claim 1 as fuel or fuel additive.

21. A fuel or fuel composition comprising the ether produced by the method of claim 1 as fuel component, optionally blended with one or more of gasoline and gasoline-ethanol blends, kerosene, diesel, biodiesel (a non-petroleum-based diesel fuel consisting of short chain alkyl (methyl or ethyl) esters, made by transesterification of vegetable oil), Fischer-Tropsch liquids, diesel-biodiesel blends and green diesel (a hydrocarbon obtained by hydrotreating biomass derived oils, fats, greases or pyrolysis oil; containing no sulphur and having a cetane number of 90 to 100) and blends of diesel and/or biodiesel with green diesel and with other derivatives of furan and tetrahydrofuran.

22. A fuel or fuel composition as claimed in claim 21, based on the ether of HMF and dihydropyran.

23. A fuel or fuel composition as claimed in claim 21, based on the ether of HMF and isobutene.
